(19) United States
(12) Patent Application Publication
(10) Pub. No.: US 2008/0214675 A1
(43) Pub. Date: Sep. 4, 2008

(54) Hydroxybenzoic Acid Amides and the Use Thereof For Masking Bitter Taste

(75) Inventors: Jakob Ley, Holzminden (DE); Heinz-Jurgen Bertram, Holzminden (DE); Gunter Kindel, Hoxter (DE); Gerhard Krammer, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/574,277

(22) PCT Filed: Aug. 2, 2005
(86) PCT No.: PCT/EP05/53764

§ 371 Date: Nov. 13, 2007

(30) Foreign Application Priority Data
Aug. 27, 2004 (DE) 10 2004 041 496.3

(51) Int. Cl. A61K 31/166; C07C 233/73; A23L 1/22

(52) U.S. Cl. 514/622; 564/179; 426/538

(57) ABSTRACT

The use is described of hydroxybenzoic acid amides having formula (I)
wherein
R¹ to R⁵ mutually independently denote hydrogen, hydroxy, methoxy or ethoxy, with the proviso that at least one of the radicals R¹ to R⁵ denotes hydroxy.
and
R⁶ denotes hydrogen, methyl or ethyl
and
n denotes 1 or 2,
their salts and mixtures thereof, to mask or reduce the unpleasant flavour impression of an unpleasantly tasting substance and/or to strengthen the sweet flavour impression of a sweet substance.

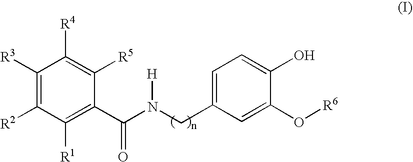

The invention relates to the use of certain hydroxybenzoic acid amides, their salts and mixtures thereof to mask or reduce unpleasant flavour impressions, particularly bitter, astringent and/or metallic flavour impressions, and/or to strengthen the sweet flavour impression of a sweet substance. Certain of these hydroxybenzoic acid amides are novel. The invention also relates to certain preparations which contain an effective content of the hydroxybenzoic acid amides, their salts or mixtures thereof. Finally the invention relates to processes for producing hydroxybenzoic acid amides for use according to the invention.

Foodstuffs or beverages commonly contain various bitter principles which although on the one hand desirable and characteristic in moderation (e.g. caffeine in tea or coffee, quinine in bitter lemon drinks, hop extracts in beer), can on the other hand also severely detract from the value (e.g. flavonoid glycosides and limonoids in citrus juices, the bitter aftertaste of many artificial sweeteners such as aspartame or saccharine, hydrophobic amino acids and/or peptides in cheese).

In order to reduce the natural content of bitter principles, a subsequent treatment is therefore often necessary, by extraction for example, as in the decaffeination of tea or coffee, or by an enzymatic process; e.g. the treatment of orange juice with a glycosides to destroy the bitter naringin or the use of special peptidases in the ripening of cheese. This treatment places a strain on the product, generates waste products and also gives rise to solvent residues and other residues (enzymes) in the products, for example.

It is therefore desirable to find substances which can effectively suppress or at least reduce unpleasant flavour impressions, in particular bitter, astringent and/or metallic flavour impressions.

The suppression of the bitter taste of many pharmaceutical active ingredients is particularly important, since the willingness of patients, particularly patients who are sensitive to bitter principles, such as children, to take the preparation orally can be significantly increased in this way. Many pharmaceutical active ingredients, for example aspirin, salicin, paracetamol, ambroxol or quinine, to name just a very small selection by way of clarification, have a marked bitter, astringent and/or metallic taste and/or aftertaste.

Although some substances are known which can partially suppress the bitter taste, many of them are severely limited in their application.

In U.S. Pat. No. 5,637,618 a bitter taste is reduced using lactisole [2O-(4-methoxyphenyl)lactic acid]. However, this inhibitor also strongly inhibits the sweet flavour impression (cf U.S. Pat. No. 5,045,336), which severely limits its applicability.

2,4-Dihydroxybenzoic acid potassium salt is described in U.S. Pat. No. 5,643,941 (table column 3, line 18) as a masking agent for the bitter taste of potassium chloride, but it cannot suppress the taste of caffeine, for example.

According to GB 2,380,936 the taste of bitter pharmaceuticals is suppressed with ginger extracts. However, the strong aroma impression and/or the pungency which is commonly to be found in ginger extracts or active ingredients obtained from them is unsuitable for many applications.

Neohesperidin dihydrochalcone likewise has a bitterness-reducing effect, but it is primarily a sweetener (cf. Manufacturing Chemist 2000, July edition, p. 16-17), which also has an intrusive effect in non-sweet applications.

Whilst flavour-modifying properties am described in U.S. Pat. No. 5,580,545 for some flavones (2-phenyl chrom-2-en-4-ones), a bitterness-reducing or suppressing action has not been found.

US 2002 177,576 describes the suppression of a bitter taste by nucleotides, for example cytidine-5′-monophosphates (CMP). The highly polar compounds, which can therefore only be used in highly polar solvents, are only of very limited use in many fatty foodstuffs, however. In addition, the availability of such substances is extremely limited due to their expensive chemical synthesis.

US 2002 188,019 describes hydroxyflavanones as effective masking agents for bitter tastes, but they are only obtainable synthetically with difficulty and are not available in larger amounts at a reasonable cost.

The sodium salts sodium chloride, sodium citrate, sodium acetate and sodium lactate have a bitterness-masking effect against many bitter principles (e.g. Nature, 1997, vol. 387, p. 563); however, the intake of large amounts of sodium ions can lead to heart and circulatory diseases, for example. Disadvantageously, a significant bitterness-masking effect also sets in only with relatively high sodium concentrations (from about 0.1 M), which corresponds for example to a generally unacceptably high content of about 0.6 wt. % of NaCl in the final application (cf R. S. J. Keast, P. A. S. Breslin and G. K. Beauchamp, Chimie 2001, 55(5), 441-447).

WO 00/21390 describes polyglutamic acid as a bitterness-masking agent; relatively high concentrations of around 1 wt. % are needed in this case.

A lipoprotein consisting of β-lactoglobulin and phosphatide acid likewise has a bitterness-masking effect (EP-A 635 218). Such polymers are difficult to characterise and standardise, however, and have a pronounced soapy aftertaste.

The flavone glycoside neodiosmin [5,7-dihydroxy-2-(4-methoxy-3-hydroxyphenyl)-7-O-neohesperidosyl chrom-2-en-4-one] likewise has a bitterness-masking effect (U.S. Pat. No. 4,154,862), but it is characterised by a disaccharide radical which makes production or isolation and applicability of the substance much more difficult.

The primary object of the present invention was to find substances which are suitable for masking or reducing the unpleasant flavour impression of unpleasantly tasting substances (and which preferably have in particular a bitterness-masking effect against a large number of bitter principles), do not negatively influence other, not unpleasant, flavours, can be widely used and are easily available.

The stated object is achieved according to the invention through the use of hydroxybenzoic acid amides having formula (I)

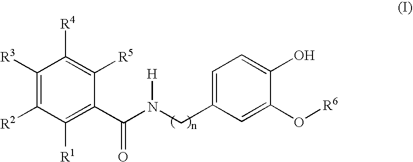

wherein

R¹ to R⁵ to mutually independently denote hydrogen, hydroxy, methoxy or ethoxy, with the proviso that at least one of the radicals R¹ to R⁵ denotes hydroxy,

and

R⁶ denotes hydrogen, methyl or ethyl

and

n denotes 1 or 2,

their salts and mixtures thereof a) to mask or reduce the unpleasant flavour impression of an unpleasantly tasting substance and/or b) to strengthen the sweet flavour impression of a sweet-tasting substance, in other words as a flavour corrector.

The use of hydroxybenzoic acid amides having the above formula (I), wherein

R¹, R³ and R⁵ mutually independently denote hydrogen or hydroxy, with the proviso that at least one of said radicals denotes hydroxy.

and

R² and R⁴ denote hydrogen,

and

R⁶ denotes hydrogen, methyl or ethyl

and

n denotes 1 or 2,

is their salts and mixtures thereof, is preferred.

The use of novel hydroxybenzoic acid amides having the above formula (I)

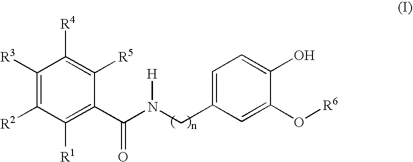

wherein

R¹ denotes hydroxy;

R³ and R⁵ mutually independently denote hydrogen or hydroxy,

and

R² and R⁴ denote hydrogen,

and

R⁵ denotes hydrogen, methyl or ethyl

and

n denotes 1 or 2,

their salts and mixtures thereof, is particularly preferred.

R⁶ here is preferably methyl or ethyl and n is preferably 1.

Unpleasantly tasting substances within the meaning of the invention are:
(a) Substances which taste bitter, astringent, sticky, dusty, dry, mealy, rancid and/or metallic and
(b) Substances which have a bitter, astringent, sticky, dusty, dry, mealy, rancid or metallic aftertaste.

The abovementioned unpleasantly tasting substances can also have other, generally not unpleasant flavour and/or odour qualities. Examples which can be cited of other, not unpleasant flavour qualities within the meaning of the present invention are, for example, spicy, umami, sweet, salty, sour, sharp, cooling, warming, burning or tingling impressions.

Substances which taste bitter, astringent, sticky, dusty, dry, mealy, rancid or metallic are, for example; xanthine alkaloids, xanthines (caffeine, theobromine, theophylline), alkaloids (quinine, brucine, strychnine, nicotine), phenolic glycosides (e.g. sailicin, arbutin), flavonoid glycosides (e.g. hesperidin, naringin), chalcone or chalcone glycosides, hydrolisable tannins (garlic or elagic acid esters of carbohydrates, e.g. pentagalloyl glucose), non-hydrolysable tannins (optionally galloylised catechins or epicatechins and oligomers thereof, e.g. proanthocyanidins or procyanidins, thearubigin), flavones (e.g. quercetin, taxifolin, myricetin), other polyphenols (γ-oryzanol, caffeic acid or esters thereof), terpenoid bitter principles (e.g. limonoids such as limonin or nomilin from citrus fruits, lupolones and humolones from hops, iridoids, secoiridoids), absinthin from wormwood, amarogentin from gentian, metallic salts (potassium chloride, sodium sulfate, magnesium sulfate), pharmaceutical active ingredients (e.g, fluoroquinolone antibiotics, paracetamol, aspirin, β-lactam antibiotics, ambroxol, propyl thiouracil [PROP], guaifenesin), vitamins (for example vitamin H, B-series vitamins such as vitamin B1, B2, B6, B12, niacin, panthotenic acid), denatonium benzoate, sucralose octaacetate, potassium chloride, magnesium salts, iron salts, aluminium salts, zinc salts, urea, unsaturated fatty acids. In particular unsaturated fatty acids in emulsions, amino acids (e.g. leucine, isoleucine, valine, tryptophane, proline, histidine, tyrosine, lysine or phenylalanine), peptides (in particular peptides with an amino acid from the group comprising leucine, isoleucine, valine, tryptophane, proline or phenylalanine at the N- or C-terminus).

Substances which have a bitter, astringent, sticky, dusty, dry, mealy, rancid or metallic aftertaste can belong for example to the group of sweeteners or sugar substitutes. Examples which can be cited include aspartame, neotame, superaspartame, saccharine, sucralose, tagatose, monellin, stevioside, thaumatin, miraculin, glycerrhizin, glycyrrhetinic acid or derivatives thereof, cyclamate or the pharmaceutically acceptable salts of the abovementioned compounds.

Sweet-tasting substances (including plant extracts) can be, for example, sweet-tasting carbohydrates (e.g. sucrose, trehalose, lactose, maltose, melicitose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde), sugar alcohols (e.g. erythritol, threitol, arabitol, ribitol, xylitol, sorbitol, mannitol, duicitol, lactitol), proteins (e.g. miraculin, monellin, thaumatin, curculin, brazzein), sweeteners (e.g. MAGAP, sodium cyclamate, acesulfame K, neohesperidin dihydrochalcone, saccharine sodium salt, aspartame, superaspartame, neotame, sucralose, stevioside, rebaudioside, lugduname, carrelame, sucrononate, sucrooctate), certain sweet-tasting amino acids (glycine, D-leucine, D-threonine, D-asparagine, D-phenylalanine, D-tryptophan, L-proline), other sweet-tasting, low-molecular-weight substances (e.g hemandulcin, dihydrochalcone glycosides, glycyrrhetinic acid derivatives), extracts of liquorice (Glycyrrhizza glabra ssp.), sugar beet (Beta vulgaris ssp.), sugar cane (Saccharum officinarum ssp.), Lippia ssp. (e.g. Lippia dulcis) or Stevia ssp. (e.g. Stevia rebaudiana).

In salts of a hydroxybenzoic acid amide having formula (I) above (wherein regarding the preferred meanings of the radicals and variables the aforesaid meanings still apply) for use according to the invention, one, more than one or all hydroxyl groups of the hydroxybenzoic acid amide are deprotonated. A corresponding quantity of counter-cations is then present, these being preferably selected from the group comprising: unipositive cations from the first main and subgroup, ammonium ions, trialkyl ammonium ions, dipositive cations from the second main and subgroup and tripositive cations from the third main and subgroup, and mixtures thereof. It goes without saying that the number of hydroxyl groups in the underlying hydroxybenzoic acid amide determines the maximum degree of deprotonation and thus also the quantity of counter-cations present. If for example only two hydroxyl groups are present in total in the underlying hydroxybenzoic acid amide, complete deprotonation of the hydroxyl groups leads to a dinegative amide anion, so a corresponding number of positive charges mast be provided by the counter-cation(s).

Particularly preferred cations are Na⁺, K⁺, NH₄⁺, Ca²⁺, Mg²⁺, Al³⁺ and Zn²⁺.

The following are particularly preferably used for the purposes according to the invention:

2,4-Dihydroxybenzoic acid-N-(4-hydroxy-3-methoxybenzyl)amide (1),

2,4,6-Trihydroxybenzoic acid-N-(4-hydroxy-3-methoxybenzyl)amide (2),

2-Hydroxybenzoic acid-N-4-(hydroxy-3-methoxybenzyl)amide (3),

4-Hydroxybenzoic acid-N-(4-hydroxy-3-methoxybenzyl)amide (4),

2,4-Dihydroxybenzoic acid-N-(4-hydroxy-3-methoxybenzyl)amide monosodium salt (5),

2,4-Dihydroxybenzoic acid-N-2-(4-hydroxy-3-methoxyphenyl)ethylamide (6),

2,4-Dihydroxybenzoic acid-N-(4-hydroxy-3-ethoxybenzyl)amide (7),

2,4-Dihydroxybenzoic acid-N-(3,4-dihydroxybenzyl)amide (8)

and

2-Hydroxy-5-methoxy-N-[2-(4-hydroxy-3-methoxyphenyl)ethyl]amide(aduncamide) (9).

Novel compounds among these are compounds (1) to (8).

The structures of the novel compounds (1) to (8) and of aduncamide (9) are provided below for clarification.

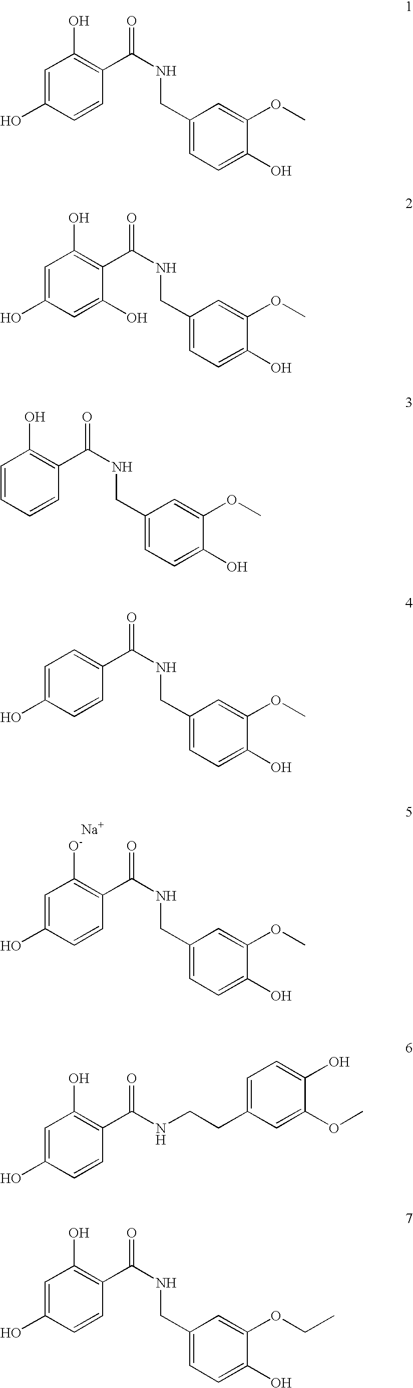

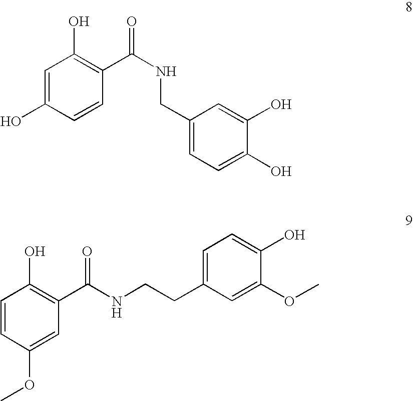

The various hydroxybenzoic acid amides and their salts for use according to the invention can naturally be used according to the invention either alone or as mixtures.

WO 03/101927A1 describes the compounds 3,4-dihydroxybenzoic acid-N-3,4-dihydroxybenzylamide and 3,4-dihydroxybenzoic acid-N-2-(3,4-dihydroxyphenyl)ethylamide as substances that in pharmaceutical preparations serve to combat amyloid-induced diseases such as e.g. Alzheimer's disease, type 2 diabetes or Parkinson's disease. However, the disclosed compounds contain two ortho-dihydroxy(dicatechol) groups, which leads to an increased instability of the compounds with regard to oxidative processes. Furthermore, no reference to the use according to the invention described here can be inferred from WO 03/101927A1.

Natural Product Letters, Vol. 2, 1993, pages 231-236 describes the (very weak) cytotoxic and antifungal action of the aduncamide isolated from Piper aduncum, 2-hydroxy-5-methoxybenzoic acid-N-[2-(4-hydroxy-3-methoxyphenyl)ethyl]amide. A use of this substance as a flavour corrector is not described, however.

EP 613,879-A1 describes the compounds 3-hydroxy-4-methoxybenzoic acid-N-(4-hydroxy-3-methoxybenzyl)amide and 4-hydroxy-3-methoxybenzoic acid-N-(4-hydroxy-3-methoxybenzyl)amide as type IV allergy-suppressing active ingredients. A use as a flavour corrector is not described, however.

WO 2004/026292 A1 discloses the compound N-(4-hydroxy-3-methoxybenzyl)-2-hydroxy-4,6-dimethoxybenzamide, but a use as a flavour corrector is not described.

J. Med. Chem., 1981, volume 24, no. 4, pages 408-428 describes a structurally related compound 2,4,6-trihydroxybenzoic acid-N-(3-hydroxy-4-methoxybenzyl)amide (referred to therein as compound no. 32), which was examined in the context of various sweeteners but which proved to be tasteless. There is however no suggestion in the cited publication that the compound described or its positional isomers could have a flavour-modulating, in particular a bitterness-masking effect, importance was attached in this study to the existence of a 3-hydroxy-4-methoxyphenyl group, which is unsuitable for use in the context of the present invention in our own studies, 2,4-dihydroxybenzoic acid-N-(3-hydroxy-4-methoxybenzyl)amide (Example 9) was prepared as a comparative compound and examined for the presence of a bitterness-masking effect (application example 1). No statistically significant change in the bitterness impression was found.

Surprisingly it was found that even in very small concentrations the hydroxybenzoic acid amides for use according to the invention can reduce or even completely suppress the unpleasant flavour impression, in particular the bitter flavour impression, of a large number of substances, in particular of methyl xanthines such as e.g caffeine, alkaloids such as e.g. quinine, flavonoids such as e.g. naringin, phenols such as e.g, salicin, inorganic salts such as potassium chloride or manganese sulfate, pharmaceutical active ingredients such as e.g. denatonium benzoate or β-lactam antibiotics, it being particularly advantageous that the hydroxybenzoic acid amides for use according to the invention have virtually no inherent flavour and do not negatively influence the other, generally not unpleasant, flavour qualities. If was likewise surprising that the hydroxybenzoic acid amides for use according to the invention also have a sweetness strengthening effect and therefore also have the ability, by simultaneously reducing the bitterness and increasing the sweet taste, to be used extremely effectively as flavour correctors.

The hydroxybenzoic acid amide, salt or mixture thereof for use according to the invention is preferably used in a food, oral care or beverage preparation or an oral pharmaceutical preparation or a cosmetic preparation for application in the head area.

A further aspect of the present invention relates to such preparations. Preparations according to the invention are used for (a) foodstuffs, (b) beverages or (c) oral care or are (d) oral pharmaceutical preparations or are (e) cosmetic preparations for application in the head area.

Food, oral care or beverage preparations and cosmetic preparations for application in the head area according to the invention preferably contain 0.000001 wt. % to 95 wt. %, based on the total weight of the preparation, of a hydroxybenzoic acid amide, salt or mixture thereof according to the invention.

An oral pharmaceutical preparation according to the invention preferably contains 0.000001 wt. % to 10 wt. % based on the total weight of the preparation, of a hydroxybenzoic acid amide, salt or mixture thereof according to the invention and also at least one unpleasantly tasting substance (see the definition above).

Of particular relevance are preparations according to the invention which contain at least one unpleasantly tasting substance, the amount of the unpleasantly tasting substance being sufficient, in a comparative preparation containing no hydroxybenzoic acid amide, salt or mixture thereof according to the invention but otherwise having an identical composition, to be perceived as an unpleasant taste, and the amount of the hydroxybenzoic acid amide, salt or mixture thereof according to the invention in the preparation being sufficient to sensorially mask the unpleasant flavour impression of the unpleasantly tasting substance or to reduce it in comparison to the comparative preparation.

Preparations according to the invention can take the form of a semi-finished product, a perfume, aromatic or flavouring composition or a spice mix.

Food or beverage preparations within the meaning of the invention are, for example, baked goods (e.g. bread, dry biscuits, cakes, other pastries), confectionery (e.g. chocolates, chocolate bar products, other bar products, fruit gums, hard and soft caramels, chewing gum), alcoholic or non-alcoholic drinks (e.g. coffee, tea, wine, wine-based drinks, beer, beer-based drinks, liqueurs, spirits, brandies, fruit-based soft drinks, isotonic drinks, soft drinks, nectars, fruit and vegetable juices, fruit or vegetable juice preparations), instant drinks (e.g. instant chocolate drinks, instant tea drinks, instant coffee drinks), meat products (e.g. ham, cured or uncured sausage preparations, spiced or marinated fresh or salted meat products), eggs or egg products (dried egg, egg white, egg yolk), cereal products (e.g. breakfast cereals, muesli bars, pre-fermented prepared rice products), dairy products (e.g. milk drinks, ice cream, yoghurt, kefir, cream cheese, soft cheese, hard cheese, dried milk powder, whey, butter, buttermilk, partially or completely hydrolysed milk protein-containing products), products made from soya protein or other soya bean fractions (e.g. soya milk and products made therefrom, preparations containing soya lecithin, fermented products such as tofu or tempo or products made therefrom), fruit preparations (e.g. jams, fruit sorbets, fruit sauces, fruit fillings), vegetable preparations (e.g. ketchup, sauces, dried vegetables, frozen vegetables, pre-fermented vegetables, preserved vegetables), snacks (e.g, baked or fried potato crisps or potato dough products, extruded products based on maize or peanuts), products based on fats and oils or emulsions thereof (e.g. mayonnaise, remoulade, dressings), other ready meals and soups (e.g, dried soups, instant soups, pre-fermented soups), spices, spice mixes and in particular seasonings, which are used in the snacks sector for example. The preparations within the meaning of the invention can also be used as semi-finished products for the production of other food or beverage preparations. The preparations within the meaning of the invention can also take the form of capsules, tablets (unseated and coated tablets, e.g. stomach acid-resistant coatings), pastilles, granules, pellets, solids mixtures, dispersions in the liquid phase, emulsions, powders, solutions, pastes or other swallowable or chewable preparations as food supplements.

Oral care preparations within the meaning of the invention are in particular mouth and/or tooth care products such as toothpastes, tooth gels, tooth powders, mouthwashes, chewing gums and other oral care products.

Oral pharmaceutical preparations within the meaning of the invention are preparations which take the form for example of capsules, tablets (unseated and coated tablets, e.g. stomach acid-resistant coatings), pastilles, granules, pellets, solids mixtures, dispersions in liquid phases, emulsions, powders, solutions, pastes or other swallowable or chewable preparations and which are used as prescription drugs, over-the-counter drugs or other drugs or as food supplements.

Cosmetic preparations for application in the head area are in particular those which contain an unpleasantly fasting substance and which even when applied correctly to the skin can come into contact with the oral cavity, in other words, for example—as already mentioned—cosmetic preparations for application in the head area, such as soaps, other cleansing or care products for the facial area, face creams or lotions or ointments, sunscreens, beard shampoos or conditioners, shaving foams, soaps or gels, lipsticks or other lip cosmetics or lip care products.

Other conventional active ingredients, basic substances, auxiliary substances and additives for food, oral care or beverage preparations or oral pharmaceutical preparations or cosmetic preparations in the head area can conventionally be included in quantities of 5 to 99.999999 wt. %, preferably 10 to 80 wt. %, based on the total weight of the preparation. The preparations can also contain water in a quantity of up to 99.999999 wt. %, preferably 5 to 80 wt. %, based on the total weight of the preparation.

The preparations according to the invention, containing one or more of the hydroxybenzoic acid amides or their salts or mixtures thereof for use according to the invention are produced in accordance with a preferred embodiment by incorporating the hydroxybenzoic acid amides or their salts or mixtures thereof for use according to the invention without solvent, as a solution or in the form of a mixture with a solid or liquid carrier in a food, oral care or beverage or oral pharmaceutical base preparation. Preparations according to the invention in the form of a solution can advantageously also be converted to a solid preparation by spray drying.

According to a further preferred embodiment, in order to produce preparations according to the invention, the hydroxybenzoic acid amides or their salts or mixtures thereof for use according to the invention and optionally other constituents of the preparation according to the invention are incorporated, even in advance, into emulsions, into liposomes, e.g, starting from phosphatidyl cholin, into microspheres, into nanospheres or into capsules, granules or extruded products made from a suitable matrix for foodstuffs and beverages, e.g. from starch, starch derivatives, cellulose or cellulose derivatives (e.g. hydroxypropyl cellulose), other polysaccharides (e.g. alginate), natural fats, natural waxes (e.g. beeswax, carnauba wax) or from proteins, e.g. gelatine. in a further preferred production process, the hydroxybenzoic acid amides or their salts or mixtures thereof are first complexed with one or more suitable complexing agents, for example with cyclic polysaccharides such as cyclofructans, cyclodextrins or cyclodextrin derivatives, preferably β-cyclodextrin or γ-cyclodextrin, and used in this complexed form.

A preparation according to the invention is particularly preferred wherein the matrix is chosen such that the hydroxybenzoic acid amides undergo a delayed release from the matrix, such that a lasting effect is achieved.

As other constituents for food or beverage preparations according to the invention, conventional basic substances, auxiliary substances and additives for foodstuffs or beverages can be used, e.g. water, mixtures of fresh or processed, plant-based or animal-based basic substances or raw materials (e.g. raw, roast, dried, fermented, smoked and/or boiled meat, bone, gristle, fish, vegetables, fruit, herbs, nuts, vegetable or fruit juices or pastes or mixtures thereof), digestible or indigestible carbohydrates (e.g. sucrose, maltose, fructose, glucose, dextrin, amylose, amylopectin, inulin, xylan, cellulose), sugar alcohols (e.g. sorbitol), natural or hydrogenated fats (e.g. tallow, lard, palm oil, coconut butter, hydrogenated vegetable fat), oils (e.g. sunflower oil, groundnut oil, maize oil, olive oil, fish oil, soya oil, sesame oil), fatty adds or salts thereof (e.g, potassium stearate), proteinogenic or non-proteinogenic amino acids and related compounds (e.g. taurin), peptides, native or processed proteins (e.g. gelatine), enzymes (e.g. peptidases), nucleic acids, nucleotides, flavour correctors for unpleasant flavour impressions, flavour correctors for other, generally not unpleasant flavour impressions, flavour-modulating substances (e.g. inositol phosphate, nucleotides such as guanosine monophosphate, adenosine monophosphate or other substances such as sodium glutamate or 2-phenoxypropionic acid), emulsifiers (e.g. lecithins, diacyl glycerols), stabilisers (e.g. carageenan, alginate), preservatives (e.g. benzoic acid, sorbic acid), antioxidants (e.g. tocopherol, ascorbic acid), chelating agents (e.g. citric acid), organic or inorganic acidulators (e.g. malic acid, acetic acid, citric acid, tannic acid, phosphoric acid), additional bitter principles (e.g. quinine, caffeine, limonin, amarogentin, humolones, lupolones, catechins, tannins), sweeteners (e.g. saccharine, cyclamate, aspartame, neotame), mineral salts (e.g. sodium chloride, potassium chloride, magnesium chloride, sodium phosphates), substances preventing enzymatic browning (e.g. sulfite, ascorbic acid), essential oils, plant extracts, natural or synthetic dyes or coloured pigments (e.g. carotinoids, flavonoids, anthocyans, chlorophyll and derivatives thereof), herbs, trigeminally active substances or plant extracts containing trigeminally active substances, synthetic, natural or nature-identical aromatic substances or perfumes and odour correctors.

Tooth care products (as a basis for oral care preparations), which contain the hydroxybenzoic acid amides, salts or mixtures thereof for use according to the invention, generally contain an abrasive system (grinding or polishing agent), such as e.g. silicas, calcium carbonates, calcium phosphates, aluminium oxides and/or hydroxyl apatites, surface-active substances, such as e.g, sodium lauryl sulfate, sodium lauryl sarcosmate and/or cocamidopropyl betaine, humectants, such as e.g. glycerol and/or sorbitol, thickeners, such as e.g. carboxymethyl cellulose, polyethylene glycols, carrageenans and/or Laponite®, sweeteners, such as e.g. saccharine, flavour correctors for unpleasant flavour impressions, flavour correctors for other, generally not unpleasant flavour impressions, flavour-modulating substances (e.g. inositol phosphate, nucleotides such as guanosine monophosphate, adenosine monophosphate or other substances such as sodium glutamate or 2-phenoxypropionic acid), cooling agents, such as e.g. menthol or menthol derivatives, stabilisers and active ingredients, such as e.g, sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, cetyl pyridinium chloride, aluminium lactate, potassium citrate, potassium nitrate, potassium chloride, strontium chloride, hydrogen peroxide, aromas and/or sodium bicarbonate or odour correctors.

Chewing gums (as a further example of oral care preparations), which contain hydroxybenzoic acid amides, salts or mixtures thereof for use according to the invention, generally comprise a chewing gum base, in other words a chewing compound that becomes plastic when chewed, sugars of various types, sugar substitutes, sweeteners, sugar alcohols, flavour correctors for unpleasant flavour impressions, flavour correctors for other, generally not unpleasant flavour impressions, flavour-modulating substances (e.g. inositol phosphate, nucleotides such as guanosine monophosphate, adenosine monophosphate or other substances such as sodium glutamate or 2-phenoxypropionic acid), humectants, thickeners, emulsifiers, aromas and stabilisers or odour correctors.

All other conventional active ingredients, basic substances, auxiliary substances and additives for oral pharmaceutical preparations can be used as constituents for oral pharmaceutical preparations according to the invention. In particular, unpleasantly tasting orally formulatable pharmaceutical active ingredients can also be used as active ingredients. The active ingredients, basic substances, auxiliary substances and additives can be converted to the oral administration forms by methods known per se. This is commonly done using inert, non-toxic, pharmaceutically suitable auxiliary substances. These include inter alia carriers (e.g, microcrystalline cellulose), solvents (e.g. liquid polyethylene glycols), emulsifiers (e.g. sodium dodecyl sulfate), dispersants (e,g. polyvinyl pyrrolidone), synthetic and natural biopolymers (e.g. albumin), stabilisers (e.g. antioxidants such as ascorbic acid), eyes (e.g. inorganic pigments such as iron oxides) and odour correctors and flavour correctors not affecting the bitter taste.

The preparations according to the invention can preferably also contain an aroma composition to round off and to improve the flavour and/or smell of the preparation. Suitable aroma compositions include, for example, synthetic, natural or nature-identical aromatic, perfume and flavouring substances and suitable auxiliary substances and carriers. It is regarded as being particularly advantageous if a bitter or metallic flavour impression deriving from aromatic substances or perfumes contained in the preparations according to the invention can be masked or reduced, thereby improving the overall aroma or flavour profile.

Preparations according to the invention in the form of semi-finished products can be used to mask or reduce the unpleasant flavour impression of finished preparations produced using the semi-finished preparation.

Preparations according to the invention which are used as semi-finished products generally contain 0.0001 wt. % to 95 wt. %, preferably 0.001 to 80wt. %, but in particular 0.01 wt. % to 50 wt. %, based on the overall weight of the preparation, of hydroxybenxoic acid amides, their salts or mixtures thereof for use according to the invention. Preparations according to the invention in the form of semi-finished products can be used to mask or reduce the unpleasant flavour impression of finished preparations produced using the semi-finished preparation.

In a particularly preferred embodiment of the invention, the hydroxybenzoic acid amides according to the invention, their salts or mixtures thereof are used in the preparations according to the invention in combination with at least one further substance to modify, mask or reduce the unpleasant flavour impression of an unpleasantly tasting substance. A particularly effective masking can be obtained in this way. In particular, the combination of the hydroxybenzoic acid amides for use according to the invention with other flavour correctors for unpleasant, in particular bitter, flavour impressions is preferred.

The other flavour correctors can be chosen from the following list, without restricting the scope of the invention: nucleotides (e.g. adenosine-5′-monophosphate, cytidine-5′-monophosphate), lactisole, sodium salts (e.g. sodium chloride, sodium lactate, sodium citrate, sodium acetate, sodium gluconate), hydroxyflavanones (e.g. eriodictyol, homoeriodictyol or the sodium salts thereof according to EP 1 258 200) amino acids or mixtures of whey proteins with lecithins.

The optionally novel hydroxybenzoic acid amides having formula (I) for use according to the invention can be produced in a process according to the invention comprising the following steps:

Reacting a compound having formula (II)

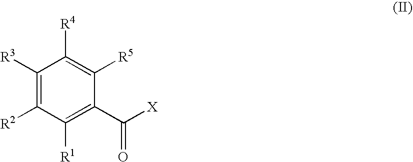

wherein

R¹ to R⁵ have the meaning specified above (preferably one characterised as being preferred) and

X is a hydroxyl group, alkyloxy or alkenyloxy group, an (optionally substituted) aryloxy group, an N-heterocyclyloxy group, an N-heterocyclyl group, a halogen atom, an (optionally substituted) sulfur atom, an —O—N group having a (poly)substituted nitrogen atom or an R—C(O)—O— group, wherein R denotes an alkyl or alkenyl radical, preferably a halogen atom, a nitro-substituted aryloxy group, an aromatic sulfonyloxy group, an N-heterocyclyl group or a cyclic (optionally substituted) hydroxylamine or another carboxyl-activating group, particularly preferably chlorine, bromine, the p- or o-nitrophenyloxy group, p-toluene sulfonyloxy group, N-imidazolyl group, N-benzotriazolyl group, N-oxyphthalimide group, N-oxybenzotriazole group or N-oxysuccinimide group,

with an amine having formula (III)

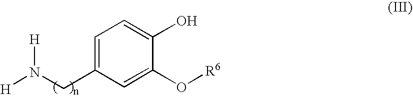

which can also take the form of its ammonium salt,

wherein R⁶ and n have the meanings specified above (preferably one characterised as being preferred),

optionally with the help of an acid scavenger or a base and/or a condensing aid,

in pure form or in a solvent or solvent blend

Optionally then processing the crude product (e.g. by methods known per se) and optionally purifying the (processed) crude product by, for example, partition, ion-exchange or gel chromatography, optionally fractional distillation, sublimation, vapour distillation, optionally fractional crystallisation, membrane processes or other customary methods.

The method is illustrated by means of the diagram below:

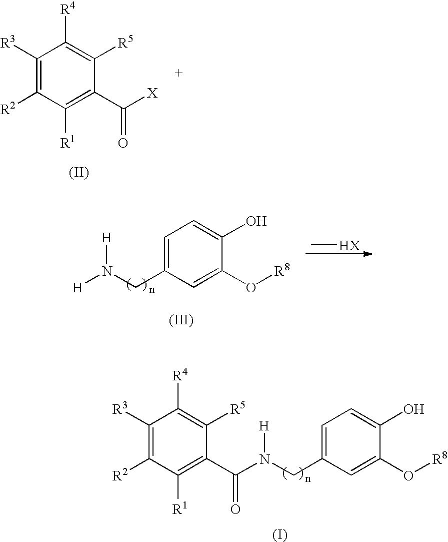

2-Hydroxy-, 3-hydroxy-, 4-hydroxy-, 2,3-dihydroxy-, 2,4-dihydroxy-, 2,5-dihydroxy-, 2-hydroxy-5-methoxy-, 2,6-dihydroxy-, 3,4-dihydroxy-, 3,5-dihydroxy-, 2,3,4-trihydroxy-, 2,3,5-trihydroxy-, 2,3,6-trihydroxy-, 2,4,5-trihydroxy-, 2,4,6-trihydroxy-, 3,4,5-trihydroxybenzoic acid or their protected derivatives, but preferably 2-hydroxy-, 4-hydroxy-, 2,4-dihydroxy- or 2,4,6-trihydroxybenzoic acid or their protected derivatives, can be used as the compound having formula (II).

4-Hydroxy-3-methoxybenzylamine, 4-hydroxy-3-ethoxybenzylamine: 2-(4-hydroxy-3-methoxyphenyl)ethylamine or 3,4-dihydroxybenzylamine are used as amines having formula (III). The ammonium salts thereof can also be used.

As acid scavengers or bases, alkali metal hydroxides (e.g. NaOH), alkali metal carbonates (e,g. Na₂CO₃ or NaHCO₃) alkaline-earth metal hydroxides (e.g. Mg(OH)₂, Ba(OH)₂), alkaline-earth metal oxides (e.g. CaO) or alkaline-earth metal carbonates (e.g. CaCO₃), ammonia, aliphatic amines (e.g. triethylamine or diisopropylamine) or heterocyclic amines (e.g. pyridine or 4-(N,N-dimethylamino)pyridine) or basic inorganic or organic ion exchangers can be used.

A dehydrating system can be used as the condensing aid, for example an activated molecular sieve, a concentrated acid or a carbodiimide. The reaction in the presence of carbodiimides is advantageously performed in a solvent or solvent blend. The reactions are preferably performed with non-polar carbodiimides, for example N,N′-dicyclohexylcarbodiimide, in ethers, in particular diethyl ether, dioxanes, tetrahydrofuran or tert-butyl methyl ether, or in esters, for example ethyl acetate, or in ketones, for example acetone.

Otherwise the following are preferred as the solvent or solvent blend: water, low alcohols (ethanol, methanol), acetone, 1,4-dioxane, tetrahydrofuran, tert-butyl methyl ether, aliphatic esters of aliphatic alcohols (such as e.g. ethyl acetate), chlorine-containing solvents (e.g. chloroform) and aromatic solvents (e.g. benzene, toluene).

### EXAMPLES

The examples serve only to clarify the invention, without restricting its scope.

### Example 1

### 2,4-Dihydroxybenzoic acid-N-(4-hydroxy-3-methyoxybenzyl)amide

2,4-Dihydroxybenzoic acid (3.08 g, 20 mmol), N-hydroxysuccinimide (2.31 g, 20 mmol) and N,N′-dicyclohexylcarbodiimide (4.12 g, 20 mmol) are placed under nitrogen in a 100 ml flask and dissolved in 1,4-dioxan (50 ml) whilst stirring. The mixture, which turns cloudy, is stirred for 16 hours at 20-25° C. The deposit is filtered off and the filtrate is poured directly into a solution of 4-hydroxy-3-methoxybenzylamine hydrochloride (3.86 g, 20.4 mmol) and sodium hydrogen carbonate (1.68 g, 20 mmol) in water (20 ml). The mixture is heated to 50° C. under nitrogen and stirred at this temperature for approximately 1 hour. After being cooled, the mixture is acidified with dilute hydrochloric acid (10%) and extracted with ethyl acetate (3×30 ml). The combined ethyl acetate phases are washed with saturated NaCl solution (30 ml), dried over Na₂SO₄, filtered and concentrated by evaporation at 40° C. in vacuo (crude 5.8 g). 2.32 g of the crude product are saponified with 10 ml of 25% sodium hydroxide solution for 1 hour at 40° C. and the mixture is then acidified with dilute hydrochloric acid. The precipitating product is extracted with a nutsch filter, washed with water and dried at 40° C./0.1 mbar.

Yield 1.49 g (extrapolated to 64%) of colourless crystals.

HPLC-MS (RP-18 phase, H₂O/acetonitrile from 95:5 to 0:100 in 30 min, then 15 min isocratic, APCI+): RT 16.6 min, m/z=289.87 (100%, [M+H]⁺), >95%.

HRMS: calculated for C₁₅H₁₅NO₅ 289.0950, found 289.0927.

¹H-NMR (400 MHz, CD₃OD, internal standard TMS): δ=7.62 (1H, d, J=8.7 Hz, H-6), 6.93 (1H, d, J=1.8 Hz, H-2′), 6.78 (1H, dd, J=8.1 Hz, J=1.9 Hz, H6′), 6.75 (1H, d, J=8.1 Hz, H-5′), 6.32 (1H, dd, J=8.7 Hz, J=2.4 Hz, H-5), 6.29 (1H, d, J=2.4 Hz, H-3), 4.45 (2H, bs, H-7′) 3.83 (3H, s, OCH₃) ppm.

¹³C-NMR (100 MHz; CD₃OD, internal standard TMS): δ=171.06 (C, C-7), 163.82 (C, C-2), 163.52 (C, C-4), 149.05 (C, C-3′) 146.82 (C, C-4′), 131.70 (C, C-1′), 130.27 (CH, C-6), 121.38 (CH, C-6′), 116.16 (CH, C-5′), 112.47 (CH, C-2′), 108.32 (C, C-1), 108.48 (CH, C-5), 103.94 (CH, C-3), 56.38 (CH₃, OCH₃), 43.80 (CH₂, C-7′) ppm.

### Example 2

### 2,4,6-Trihydroxybenzoic acid-N-(4-hydroxy-3-methoxybenzyl)amide

Starting from 2,4,6-trihydroxybenzoic acid (3.76 g, 20 mmol) 2.74 g (45% of theoretical) of the desired compound were able to be obtained as colourless crystals using the instructions given in Example 1.

HPLC-MS (RP-18 phase, H₂O/MeOH from 50:50 to 5:95 in 15 min, then 15 min isocratic, APCI+): RT 17.07 min, m/z=305.91 (100%, [M+H]⁺), >95%.

HRMS: caiculaled for C₁₅H₁₅NO₆ 305.0899, found 305.0903.

¹H-NMR (400 MHz, CD₃OD, internal standard TMS): δ=6.93 (1H, d, J=1.6 Hz, H-2′) 6.79 (1H, dd, J=8.0 Hz, J=1.7 Hz, H6′), 6.76 (1H, dd, J=8.0 Hz, J=0.5 Hz, H-5′), 5.85 (2H, s, H-3, H-5), 4.45 (2H, bs, H-7′), 3.84 (3H, s, OCH₃) ppm.

¹³C-NMR (100 MHz, CD₃OD, internal standard TMS): δ=171.85 (C, C-7). 163.48 (C, C-3, C-6), 163.20 (C, C-4), 149.14 (C, C-3′), 146,92 (C, C-4′), 131.31 (C, C-1′), 121.31 (CH, C-6′), 116.28 (CH, C-5′), 112.35 (CH, C-2′), 96.99 (C, C1), 96.02 (CH, C-3, C-5), 56.38 (CH₃, OCH₃), 43.67 (CH₂, C-7′) ppm.

### Example 3

### 2-Hydroxybenzoic acid-N-(4-hydroxy-3-methoxybenzyl)amide

Starting from 10 mmol of 2-hydroxy benzoic acid, after purifying the crude product by chromatography on silica gel with the eluent n-hexane/ethyl acetate 1:1 to 1:2 (v/v), 1.87 g (68% of theoretical) of the desired compound were able to be obtained as colourless crystals, using the instructions given in Example 1.

HPLC-MS (RP-18 phase, H₂O/acetonitrile from 100:0 to 0:100 in 60 min, APCI+): RT 15.1 min, m/z=273 90 (100%, [M+H]⁺), >95%.

HRMS: calculated for C₁₅H₁₅NO₄ 273.1001, found 273.0979.

¹H-NMR (400 MHz, d₆-DMSO, internal standard TMS): δ=9.26 (1H, t, J≈6 Hz, NH), 8.89 (1H, bs, OH), 7.88 (1H, ddd, J=7.9 Hz, J=1.7 Hz, J=0.4 Hz, H-6), 7.39 (1H, ddd, J=8.3 Hz. J=7.2 Hz, J=1.7 Hz, H-4), 6.92 (1H, bs, H-2′), 6.89 (1H, dd, J=8.3 Hz, J=1.2 Hz, H-3), 6.88 (1H, ddd, J=8.0 Hz, J=7.3 Hz, J=1.2 Hz, H-5), 6.73 (2H, m, H-5′, H-6′), 4.40 (2H, bd, J=5.7 Hz, H-7′), 3.84 (3H, s, OCH₃) ppm.

¹³C-NMR (100 MHz; d₆-DMSO, internal standard TMS): δ=163.68 (C, C-7), 160.00 (C), 147.37 (C), 145.52 (C), 133.59 (C, C-4), 129.58 (C), 127.68 (CH, C-6), 119.83 (CH), 118.48 (CH), 117.30 (CH), 115.19 (CH, C-5′) 115.19 (C), 111.87 (CH, C-2′), 55.49 (CH₃, OCH₃), 42.13 (CH₂, C-7′) ppm.

### Example 4

### 4-Hydroxybenzoic acid-N-(4-hydroxy-3-methoxybenzyl)amide

Starting from 10 mmol of 4-hydroxybenzoic acid, after purifying the crude product by chromatography on silica gel with the eluent n-hexane/ethyl acetate 1:1 (v/v), 1 g (37% of theoretical) of the desired compound was able to be obtained as colourless crystals, using the instructions given in Example 1.

HPLC-MS (RP-18 phase, H₂O/acetonitrile from 100:0 to 0:100 in 60 min, APCI+): RT 15.1 min, m/z=273.92 (66%, [M+H]⁺), 546.54 ([2M+H]⁺) >95%.

HRMS: calculated for C₁₅H₁₅NO₄ 273.1001, found 273.0996.

¹H-NMR (400 MHz, d₆-DMSO, internal standard TMS): 8.63 (1H, bd, J=5.8 Hz, NH), 7.74 (2H, dm, J=8.5 Hz, H-2, H-6), 6.88 (1H, m, H-2′), 6.78 (2H, dm, J=8.8 Hz. H-3, H-5), 6.89 (2H, m, H-5′, H-6′), 4.32 (2H, bd, J=5.9 Hz, H-7′), 3.73 (3H, s, OCH₃) ppm.

¹³C-HMR (100 MHz; d₆-DMSO, internal standard TMS): δ=165.67 (C, C-7), 159.99 (C), 147.27 (C), 145.25 (C), 130.72 (C), 129.04 (2×CH, C-2, C-6), 125.11 (C), 119.67 (CH, C-6′), 115.07 (CH, C-5′), 114.67 (2×CH, C-3, C-5), 111.75 (CH, C-2′), 55.49 (CH-₃, OCH₃), 42.13 (CH₂, C-7′) ppm.

### Example 5

### 2,4-Dihydroxybenzoic acid-N-4-hydroxy-3-methoxybenzylamide monosodium salt

The product from Example 1 (260 mg, 0.9 mmol) is completely dissolved in sodium hydroxide solution (1 mol/l, 0.9 ml), water (1 ml) and ethanol (2 ml) and stirred for 1 hour at 50° C. The mixture is concentrated by evaporation until dry at 40° C. in vacuo and the residue is ground with ethyl acetate (10 ml), the product filtered off and dried. Yield: 0.246 g

¹H-NMR (400 MHz, CD₃OD, internal standard TMS): δ=7.62 (1H, d, J=8.7 Hz, H-6), 6.95 (1H, d, J=1.8 Hz, H-2′), 6.79 (1H, dd, J=8.1 Hz, J=1.9 Hz, H6′), 6.73 (1H, d, J=8.1 Hz, H-5′), 6.10 (1H, dd, J=8.7 Hz, J=2.4 Hz, H-5), 6.03 (1H, d, J=2.4 Hz, H-3), 4.48 (2H, bs, H-7′), 3.83 (3H, s, OCH₃) ppm.

### Example 6

### 2,4-Dihydroxybenzoic acid-N-2-(4-hydroxy-3-methoxyphenyl)ethylamide

Starting from 17 mmol of 2,4-hydroxybenzoic acid and 20 mmol of 4-hydroxy-3-ethoxybenzylamine hydrochloride, after purifying the crude product by chromatography on silica gel with the eluent n-hexane/ethyl acetate 3:1 (v/v), 2.1 g (41% of theoretical) of the desired compound were able to be obtained as colourless crystals, using the instructions given in Example 1.

HPLC-MS (RP-18 phase, APCI+): RT 17.4 min, m/z=303.92 (100%, [M+H]⁺), 606.21 (1.2%, [2M+H]⁺) >95%.

HRMS: calculated for C₁₆H₁₇NO₅ 303.1107, found 303.1111.

¹H-NMR (400 MHz, CD₃OD, internal standard TMS); δ=7.54 (1H, dd, J=8.7 Hz, J=0.4 Hz, H-6), 6.81 (1H, dt, J=1.9 Hz, J=0.3 Hz, H-2′), 6.72 (1H, dd, J=8.0 Hz, J=0.3 Hz, H-3′), 6.67 (1H, ddt, J=8.0 Hz, J=1.9 Hz, J=0.5 Hz, H-6′), 6.30 (1H, dd, J=8.7 Hz, J=2.4 Hz, H-5), 6.27 (1H, dd, J=2.4 Hz, J=0.4 Hz, H-3), 3.78 (3H, d, J=0.3 Hz, O—CH₃), 3.55 (2H, m, H-8), 2.80 (2H, m, H-7) ppm.

¹³C-NMR (100 MHz; CD₃OD, internal standard TMS): δ=171.15 (C, C-7), 163.79 (C. C-2), 163.41 (C, C-4), 148.95 (C, C-3′), 146.05 (C, C-4′), 130.25 (CH, C-6), 122.33 (CH, C-6′), 116.24 (CH, C-5′), 113.52 (CH, C-2′), 108.91 (C, C-1), 103.92 (CH, C-3), 56.31 (CH₃, O—CH₃), 42.37 (CH₂, C-8), 36.30 (CH₂, C-7) ppm.

### Example 7

### 2,4-Dihydroxybenzoic acid-N-4-hydroxy-3-ethoxybenzylamide

Starring from 10 mmol of 2,4-dihydroxybenzoic acid and 12 mmol of 4-hydroxy-3-ethoxybenzxylamine hydrochloride, 1.5 g (50%) of the desired compound were able to be obtained as a colourless crystal mass using the instructions given in Example 1.

HPLC-MS (RP-18 phase, APCI+): RT 16.11 min, m/z=303.88 (100%, [M+H]⁺).

HRMS: calculated for C₁₆H₁₇NO₅ 303.1107, found 303.1118.

¹H-NMR (400 MHz, d₆-DMSO, internal standard TMS): δ=10.07 (1H, bs, OH), 8.93 (1H, bt, J=5.9 Hz, NH), 8.78 (1H, s, OH), 7.71 (1H, d, J=8.7 Hz, H-6), 6.88 (1H, d, J=1.7 Hz, H-2′), 6.72 (1H, d, J=8.0 Hz, H-5′), 6.69 (1H, dd, J=8.0 Hz, J=1.7 Hz, H-6′), 6.28 (1H, dd, J=8.7 Hz, J=2.4 Hz, H-5), 6.22 (1H, d, J=2.4 Hz, H-3), 4.34 (1H, bd, J=5.9 Hz, H-7′), 3.98 (2H, q, J=7.0 Hz, O—CH₂—CH₃), 1.31 (3H, t, J=7.0 Hz, O—CH₂—CH₃) ppm.

¹³C-NMR (100 MHz; d₆-DMSO, internal standard TMS): δ=168.83 (C, C-7), 162.24 (C, C—C-2), 161.94 (C, C-4), 146.22 (C, C-3′), 145.54 (C, C-4′), 129.64 (C, C-1′), 128.69 (CH, C-6), 119.67 (CH, C-6′), 115.04 (CH, C-5′), 113.06 (CH, C-2′), 106.72 (CH, C-5), 102.41 (CH, C-3), 63.60 (CH₂, O—CH₂—CH₃), 41.66 (CH₂, C-7′), 14.49 (CH₃, O—CH₂—CH₃) ppm.

### Example 8

### 2,4-Dihydroxybenzoic acid-N-3,4-dihydroxybenzylamide

Starting from 17 mmol of 2,4-dihydroxybenzoic acid and 20 mmol of 3,4-dihydroxybenzylamine hydrochloride, after purifying the crude product by chromatography on silica gel with the eluent n-hexane/ethyl acetate 3:1 (v/v), 1.3 g (29%) of the desired compound were able to he obtained as a colourless crystal mass, using the instructions given in Example 1.

HPLC-MS (RP-18 phase, APCI+): RT 10.34 min, m/z=276.0 (100%, [M+H]⁺).

¹H-NMR (400 MHz, d₆-DMSO, internal standard TMS): δ=10.05 (1H, s, OH), 8.93 (1H, t, J=6.0 Hz, NH), 8.85 (1H, s, OH), 8.72 (1H, s, OH), 7.72 (1H, d, J=8.8 Hz, H-6), 6.71 (1H, d, J=2.1 Hz, H-2′), 6.66 (1H, d, J=8.1 Hz, H-5′), 6.56 (1H, dd, J=3.1 Hz, J=2.1 Hz, H-6′), 6.28 (1H, dd, J=8.7 Hz, J=2.4 Hz, H-5), 6.23 (1H, d, J=2.4 Hz, H-3), 4.29 (2H, d, J=5.9 Hz, H-7′) ppm.

¹³C-NMR (100 MHz; d₆-DMSO, internal standard TMS): δ=169.04 (C, C-7), 162.52 (C, C-4), 162.13 (C-2), 145.00 (C, C-3′), 144.12 (C, C-4′), 129.90 (C, C-1′), 128.83 (CH, C-6), 118.15 (CH, C-6′), 115.23 (CH, C-5′), 114.73 (CH, C-2′), 106.89 (CH, C-5), 102.61 (CH, C-3), 41.62 (CH₂, C-7′) ppm.

### Example 9

### 2,4-Dihydroxybenzoic acid-N-3-hydroxy-4-methoxybenzylamide as a Comparative Example

Starting from 17 mmol of 2,4-hydroxybenzoic acid and 19 mmol of 3-hydroxy-4-methoxybenzylamine hydrochloride, after purifying the crude product by chromatography on silica gel with the eluent n-hexane/ethyl acetate 3:1 (v/v), 1.45 g (80% of theoretical) of the desired compound were able to be obtained as colourless crystals, using the instructions given in Example 1.

HPLC-MS (RP-18 phase, APCI+): RT 15.54 min, m/z=289.89 (100%, [M+H]⁺).

¹H-NMR (400 MHz, CD₃OD, internal standard TMS): δ=7.62 (1H, dd, J=8.7 Hz, J=0.3 Hz, H-6), 6.86 (1H, d, J=8.2 Hz, H-5′), 6.81 (1H, dd, J=2.2 Hz, J=0.3 Hz, H-2′) 6.77 (1H, ddt, J=8.2 Hz, J=2.2 Hz, J=0.6 Hz, H-6′), 6.32 (1H, ddd, J=8.7 Hz, J=2.4 Hz, J=0.3 Hz, H-5), 6.28 (1H, dd, J=2.4 Hz, J=0.3 Hz, H-3), 4.43 (2H, bs, H-7′), 3.82 (3H, s, O—CH₃) ppm.

### Application Example 1

### Bitterness Reduction in a Bitter Principle Solution

To quantify the reduction in the bitterness impression, the bitterness of a 500 ppm caffeine or salicin solution and a sample containing 500 ppm of caffeine or salicin and a varying amount of the exemplary compound was determined by a group of experts (rating 0 [not bitter] to 10 [extremely bitter]). The evaluation was made as a calculation of the reduction (in %) of the bitterness impression from the average values for the ratings of the caffeine or salicin solution and the solutions containing caffeine or salicin and the exemplary compound. 2,4-Dihydroxybenzoic acid (2,4-DHB) from U.S. Pat. No. 5,643,941 was used as a comparison.

TABLE
Bitterness of a solution containing a bitter principle and of a solution
containing a bitter principle and an exemplary compound (2,4-DHB = 2,4-
dihydroxybenzoic acid); “Testers positive” denotes the number of testers who
could detect a masking effect; the 95% confidence intervals are given as the
error; p < 0.05 denotes the significance according to the student's t-test method
(see statistics textbooks).

| | | | | Bitterness | | % reduction |
|---|---|---|---|---|---|---|
| | | Testers | | impression (1-10) | | in bitterness |
| Substance | Bitter principle | total | positive | without | with | impression |
| 100 ppm | 500 ppm | 15 | 9 | 5.1 ± 1.0 | 5.0 ± 1.0 | 3% |
| 2,4-DHB | caffeine | | | | | |
| 20 ppm | 500 ppm | 11 | 11 | 6.3 ± 1.2 | 4.5 ± 0.6 | 29%, p < 0.05 |
| Example 1 | caffeine | | | | | |
| 100 ppm | 500 ppm | 11 | 8 | 6.7 ± 0.9 | 5.1 ± 0.9 | 24%, p < 0.05 |
| Example 2 | caffeine | | | | | |
| 100 ppm | 500 ppm | 15 | 12 | 5.3 ± 1.0 | 4.1 ± 0.9 | 23%, p < 0.05 |
| Example 3 | caffeine | | | | | |
| 100 ppm | 500 ppm | 12 | 9 | 6.1 ± 1.1 | 4.5 ± 0.5 | 26%, p < 0.05 |
| Example 4 | caffeine | | | | | |
| 100 ppm | 500 ppm | 16 | 13 | 5.9 ± 0.9 | 4.1 ± 1.0 | 32%, p < 0.05 |
| Example 5 | caffeine | | | | | |
| 100 ppm | 500 ppm | 12 | 9 | 4.6 ± 1.0 | 3.7 ± 0.8 | 20%, p < 0.05 |
| Example 6 | caffeine | | | | | |
| 100 ppm | 500 ppm | | | 4.4 ± 1.1 | 3.9 ± 0.8 | 11% |
| Example 9 | caffeine | | | | | |
| (comparison) | | | | | | |

### Application Example 2

### Spray-Dried Preparation as a Semi-Finished Product for Aromatising Finished Products

| Ingredient | Use in wt. % |
|---|---|
| Drinking water | 60.8% |
| Maltodextrin from wheat | 24.3% |
| Gum arabic | 6.1% |
| 2,4-Dihydroxybenzoic | 8.8% |
| acid-N-(4-hydroxy-3-methoxybenzyl) | |
| amide (Example 1) | |

The drinking water is placed in a beaker and the maltodextrin and gum arabic are dissolved therein. Then the 2,4-dihydroxybenzoic acid-N-(4-hydroxy-3-methoxybenzyl)amide (Example 1) is emulsified into the carrier solution using a Turrax. The temperature of the spraying solution should not exceed 30° C. The mixture is then spray dried (setpoint temperature inlet: 185-195° C., setpoint temperature outlet: 70-75° C.). The spray-dried semi-finished product contains approximately 18 to 22% of the active ingredient from Example 1.

### Application Example 3

### Black Tea Preparation

| Ingredient | Use in wt. % |
|---|---|
| Black tea, Ceylon, leaf product | 94.00% |
| Semi-finished product from application example 2, | 6% |
| containing approx. 18-22% of 2,4-dihydroxybenzoic | |
| acid-N-(4-hydroxy-3-methoxybenzyl) amide (Example 1) | |

The tea and the semi-finished product are mixed together and packed into a teabag made from filter paper. In order to use it, a teabag is placed in 100 to 250 ml of boiling water and allowed to steep for 2 to 6 minutes.

### Application Example 4

### Black Tea Preparation in Combination with Homoeriodictyol Sodium Salt

| Ingredient | Use in wt. % |
|---|---|
| Black tea, Ceylon, leaf product | 94.00% |
| Semi-finished product from application example 2, | 3% |
| containing approx. 18-22% of 2,4-dihydroxybenzoic | |
| acid-N-(4-hydroxy-3-methoxybenzyl) amide (Example 1) | |
| Semi-finished product from homoeriodictyol sodium salt, | 3% |
| spray dried as in application example 2 | |

The tea and the semi-finished products are mixed together and packed into a teabag made from filter paper. In order to use it, a teabag is placed in 100 to 250 ml of boiling water and allowed to steep for 2 to 5 minutes.

### Application Example 5

### Use in a Soya Drink

The compound 2,4-dihydroxybenzoic acid-N-(4-hydroxy-3-methoxybenzyl)amide from Example 1 was pre-dissolved in ethanol and added to a soya milk from a local supermarket. The mixture was mixed with the milk aroma in a beaker.

| Ingredient | Use in wt. % |
|---|---|
| Soya milk (local supermarket) | 99.8% |
| Milk aroma | 0.1% |
| 10% 2,4-dihydroxybenzoic acid-N-(4-hydroxy-3- | 0.1% |
| methoxybenzyl)amide (Example 1) in ethanol | |

### Application Example 6

### Use in a Soya Drink in Combination with γ-Aminobutyric Acid

γ-Amincbutyric acid pre-dissolved in water and 2,4-dihydroxybenzoic acid-N-(4-hydroxy-3-methoxybenzyl)amide from Example 1 pre-dissolved in ethanol were added to a soya milk from a local supermarket. The mixture was mixed with the milk aroma in a beaker.

| Ingredient | Use in wt. % |
|---|---|
| Soya milk (local supermarket) | 99.7% |
| Milk aroma | 0.1% |
| 10% 2,4-dihydroxybenzoic acid-N-(4-hydroxy-3- | 0.1% |
| methoxybenzyl)amide (Example 1) in ethanol | |
| 1% γ-aminobutyric acid in water | 0.1% |

### Application Example 7

### Use in a Chewing Gum

| Part | Ingredient | Use in wt. % |
|---|---|---|
| A | Chewing gum base, “Jagum T” | 30.00 |
| B | Sorbitol, powdered | 39.00 |
| | Isomalt ® (Palatinit GmbH) | 9.50 |
| | Xylitol | 2.00 |
| | Mannitol | 3.00 |
| | Aspartame ® | 0.10 |
| | Acesulfame ® K | 0.10 |
| | Emulgum ® (Colloides Naturels. Inc.) | 0.30 |
| C | Sorbitol, 70% | 14.00 |
| | Glycerol | 1.00 |
| D | Aroma, containing 1% 2-hydroxybenzoic acid-N-(4- | 1 |
| | hydroxy-3-methoxybenzyl)amide (Example 3) | |

Parts A to D are mixed and compounded intensively. The crude compound can be processed into ready-to-use chewing gum in the form of thin strips, for example.

### Application Example 8

### Use in a Toothpaste

| Part | Ingredient | Use in wt. % |
|---|---|---|
| A | Demineralised water | 22.00 |
| | Sorbitol (70%) | 45.00 |
| | Solbrol ® M, sodium salt (Bayer AG, | 0.15 |
| | p-hydroxybenzoic acid alkyl ester) | |
| | Trisodium phosphate | 0.10 |
| | Saccharine, 450 times | 0.20 |
| | Sodium monofluorophosphate | 1.12 |
| | Polyethylene glycol 1500 | 5.00 |
| B | Sident 9 (abrasive silicon dioxide) | 10.00 |
| | Sident 22 S (thickening silicon dioxide) | 8.00 |
| | Sodium carboxymethyl cellulose | 0.90 |
| | Titanium dioxide | 0.50 |
| C | Demineralised water | 4.53 |
| | Sodium lauryl sulfate | 1.50 |
| D | Aroma, containing 1% 4-hydroxybenzoic acid-N-(4- | 1 |
| | hydroxy-3-methoxybenzyl)amide (Example 4) | |

The ingredients in parts A and B are each premixed and stirred together well under vacuum at 25 to 30° C. for 30 minutes. Part C is premixed and added to A and B; D is added and the mixture is stirred well under vacuum at 25 to 30° C. for 30 minutes. After releasing the vacuum, the toothpaste is ready and can be filled.

### Application Example 9

### Strengthening the Sweetness of a Sugar Solution

To quantify the strengthening of the sweetness impression, the sweetness of a 5% sucrose solution and a sample containing 5% ppm sucrose and an amount of the exemplary compound was determined by a group of experts (rating 0 [not sweet] to 10 [extremely sweet]). The evaluation was made as a calculation of the strengthening (in %) of the sweetness impression from the average values for the ratings of the sucrose solution and the solutions containing sucrose and the exemplary compound. Vanillin was used as a comparison as a typical aromatic substance described as sweet.

TABLE
Sweetness of a sucrose solution and of a solution containing
sucrose and an exemplary compound; “Testers positive” denotes the
number of testers who could detect a strengthening effect; the standard
deviations are given as the error.

| | | | | | | % |
|---|---|---|---|---|---|---|
| | | | | Sweetness | | strengthening |
| | | Testers | | impression (1-10) | | of sweetness |
| Substance | Sweetener | total | positive | without | with | impression |
| 600 ppb vanillin | 5% | 10 | 5 | 6.2 ± 1.2 | 6.0 ± 1.1 | −3% |
| | sucrose | | | | | |
| 100 ppm | 5% | 16 | 7 | 5.2 ± 1.8 | 5.6 ± 1.7 | 7.2% |
| Example 1 | sucrose | | | | | |

### Application Example 10

### Strengthening the Sweetness of a Yogurt

To quantify the strengthening of the sweetness impression, the sweetness of a low-fat (0.1%) commercial yogurt (Optiwell) containing 5% sucrose and of a sample containing 5% ppm sucrose and an amount of the exemplary compound was determined by a group of experts (rating 0 [not sweet] to 10 [extremely sweet]). The evaluation was made as a calculation of the strengthening (in %) of the sweetness impression from the average values for the ratings of the yogurt containing sucrose and the yogurt containing sucrose and the exemplary compound.

The yogurt containing Example 1 was preferred and was also described using the descriptors more fullness, less acidic, soft, creamy.

TABLE
Sweetness of a low-fat yogurt containing sucrose and of a yogurt
containing sucrose and an exemplary compound; “Testers positive”
denotes the number of testers who could detect a strengthening effect; the
standard deviations are given as the error.

| | | | | Sweetness | | % strengthening |
|---|---|---|---|---|---|---|
| | | Testers | | impression (1-10) | | of sweetness |
| Substance | Sweetener | total | positive | without | with | impression |
| 200 ppm | 5% sucrose | 16 | 12 | 4.3 ± 1.3 | 5.4 ± 1.5 | 26.5% |
| Example 1 | | | | | | |

1. A method for masking and/or reducing unpleasant flavors and/or strengthen sweet flavour impressions of a composition comprising:
adding to said composition an hydroxybenzoic acid amides having formula (I) 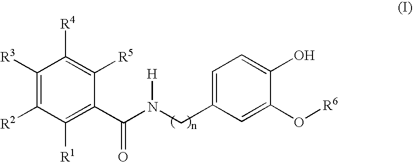
wherein
R¹ to R⁵ mutually independently denote hydrogen, hydroxy, methoxy or ethoxy, with the proviso that at least one of the radicals R¹ to R⁵ denotes hydroxy,
and R⁶ denotes hydrogen, methyl or ethyl
and n denotes 1 or 2, their salts and mixtures thereof, wherein said amide is added in an amount sufficient
a) to mask or reduce the unpleasant flavour impression of an unpleasantly tasting substance and/or
b) to strengthen the sweet flavour impression of a sweet-tasting substance.

2. A method according to claim 1, wherein
R¹, R³ and R⁵ mutually independently denote hydrogen or hydroxy, with the proviso that at least one of said radicals denotes hydroxy,
and
R² and R⁴ denote hydrogen,
and
R⁶ denotes hydrogen, methyl or ethyl
and
n denotes 1 or 2,
their salts and mixtures thereof.

3. A method according to claim 1, wherein
R¹ denotes hydroxy,
and
R³ and R⁵ mutually independently denote hydrogen or hydroxy,
and
R² and R⁴ denote hydrogen,
and
R⁶ denotes hydrogen, methyl or ethyl
and
n denotes 1 or 2, their salts and mixtures thereof.

4. A method according to claim 1 wherein a salt of said hydroxybenzoic acid amide is added to said composition:
wherein one, more than one or all hydroxyl groups of the hydroxybenzoic acid amide are deprotonated and a corresponding quantity of counter-cations is present, which are selected from the group comprising: unipositive cations from the first main and subgroup, ammonium ions, trialkyl ammonium ions, dispositive cations from the second main and subgroup and tripositive cations from the third main and subgroup, and mixtures thereof.

5. A method according to claim 4 wherein the hydroxylbenzoic acid amide salt comprises 2,4-dihydroxybenzoic acid-N-(4-hydroxy-3-methoxybenzyl)amide, 2,4,6-trihydroxybenzoic acid-N-(4-hydroxy-3-methoxybenzyl)amide, 2-hydroxybenzoic acid-N-4-(hydroxy-3-methoxybenzyl)amide, 4-hydroxybenzoic acid-N-(4-hydroxy-3-methoxybenzyl)amide, 2,4-dihydroxybenzoic acid-N-(4-hydroxy-3-methoxybenzyl)amide monosodium salt, 2,4-dihydroxybenzoic acid-N-2-(4-hydroxy-3-methoxyphenyl)ethylamide, 2,4-dihydroxybenzoic acid-N-(4-hydroxy-3-ethoxybenzyl)amide, 2,4-dihydroxybenzoic acid-N-(3,4-dihydroxybenzyl)amide or 2-hydroxy-5-methoxy-N-[2-(4-hydroxy-3-methoxyphenyl)ethyl]amide.

6. A method according to claim 1 wherein said composition comprises a food, oral care composition, beverage preparation, an oral pharmaceutical preparation, or a cosmetic preparation.

7. Hydroxybenzoic acid amides having formula (I) 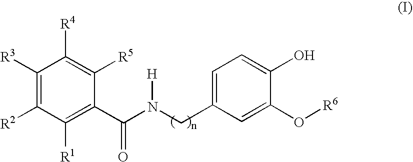
wherein
R¹ denotes hydroxy,
and
R³ and R⁵ mutually independently denote hydrogen or hydroxy,
and
R² and R⁴ denote hydrogen,
and
R⁶ denotes hydrogen, methyl or ethyl
and
n denotes 1 or 2,
their salts and mixtures thereof.

8. A food, oral care, beverage, or cosmetic preparation for application in the head area containing 0.000001 wt. % to 95 wt. %, based on the total weight of the preparation, of a hydroxybenzoic acid amide, a salt or mixture thereof as defined in claim 7.

9. An oral pharmaceutical preparation containing 0.000001 wt. % to 10 wt. %, based on the total weight of the preparation, of a hydroxybenzoic acid amide, a salt or mixture thereof as defined in claim 7 and at least one unpleasantly tasting substance.

10. A preparation according to claim 8, comprising at least one unpleasantly tasting substance, the amount of said unpleasantly tasting substance being sufficient, in a comparative preparation containing no hydroxybenzoic acid amide, salt or mixture thereof but otherwise having an identical composition, to be perceived as an unpleasant taste, and the amount of the hydroxybenzoic acid amide, salt or mixture thereof in the preparation being sufficient to sensorially mask the unpleasant flavour impression of said unpleasantly tasting substance or to reduce it in comparison to the comparative preparation.

11. Preparation according to claim 8, comprising at least one sweet-tasting substance, the amount of said sweet-tasting substance being sufficient, in a comparative preparation containing no hydroxybenzoic acid amide, salt or mixture thereof but otherwise having an identical composition, to be perceived as a sweet taste, and the amount of the hydroxybenzoic acid amide, salt or mixture thereof in the preparation being sufficient to sensorially strengthen the sweet flavour impression of the sweet-tasting substance or to strengthen it in comparison to the comparative preparation.

12. Preparation according to claim 8, in the form of a semi-finished product, a perfume, aromatic or flavouring composition or a spice mix.

13. Preparation according to claim 8, further comprising at least one further substance for modifying, masking or reducing an unpleasant flavour impression of an unpleasantly tasting substance.

14. A process for producing a hydroxybenzoic acid amide having formula (I) 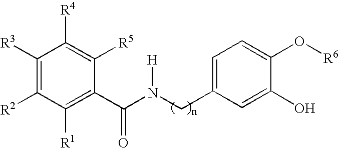
comprising
reacting under amide-forming conditions a compound having formula (II) 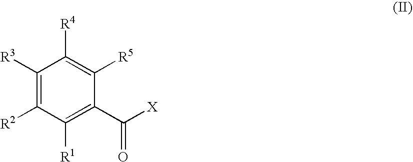
wherein
R¹ to R⁵ in formula (I) and (II) have the same meaning given above and
X is a hydroxyl group, alkyloxy or alkenyloxy group, an (optionally substituted) aryloxy group, an N-heterocyclyloxy group, an N-heterocyclyl group, a halogen atom, an (optionally substituted) sulfur atom, an —O—N group having a (poly)substituted nitrogen atom or an R—C(O)—O— group, wherein R denotes an alkyl or alkenyl radical, preferably a halogen atom, a nitro-substituted aryloxy group, an aromatic sulfonyloxy group, an N-heterocyclyl group or a cyclic (optionally substituted) hydroxylamine or another carboxyl-activating group, particularly preferably chlorine, bromine, the p- or o-nitrophenyloxy group, p-toluene sulfonyloxy group, N-imidazolyl group, N-benzotriazolyl group, N-oxyphthalimide group, N-oxybenzotriazole group or N-oxysuccinimide group,
with an amine having formula (III) 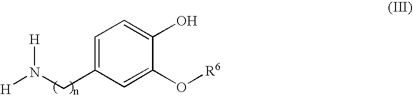
which can also take the form of its ammonium salt,
R⁶ and n having the meanings given above.
